(19) United States
(12) Patent Application Publication
(10) Pub. No.: US 2021/0330706 A1
(43) Pub. Date: Oct. 28, 2021

(54) Children Blood Transfusion and Thymus Hormonal Therapy as Antiviral and Antibacterial Compounds

(71) Applicant: Kareem Issa Batarseh, Fairfax Station, VA (US)

(72) Inventor: Kareem Issa Batarseh, Fairfax Station, VA (US)

(21) Appl. No.: 17/300,085

(22) Filed: Mar. 5, 2021

Related U.S. Application Data
(60) Provisional application No. 63/100,461, filed on Mar. 13, 2020.

(51) Int. Cl. A61K 35/16; A61K 38/22; A61K 38/32; A61K 38/08; A61K 45/06

(52) U.S. Cl. CPC A61K 35/16; A61K 38/2292; A61K 9/0019; A61K 38/08; A61K 45/06; A61K 38/32

(57) ABSTRACT

The present invention describes compounds that are safe, nontoxic, noncorrosive, and nonirritating and methods thereof that are antibacterial and antiviral (encompassing cancer as well or chemotherapy) for the prevention and treatment of individuals suffering from infectious diseases. The compounds of the instant invention can also be administered to those individuals who are in need of such therapy such as those who have thymus diseases or those who are old enough that the thymus gland does not function anymore (thymus involution). This method can also be employed in healthy subjects as a preventive measure against such infections. The method relies on administering to infected patient healthy whole blood or healthy plasma transfusion procured from healthy children where it is permissible by law (emphasis added) or from healthy adults ages 18 to 20 years. This as a result will endogenously regenerate the thymus gland via the process of thymopoiesis to produce mature T cells, which are the most important cells in adaptive immunity for combating bacterial and viral infections. The compounds of the instant invention can be those, but not limited to, hormones secreted by the thymus gland and their synthetic counterparts, a combination of them, their active compounds, their progenitors, and their stimulating and mediated compounds and their mixtures thereof all can be used in the instant invention.

This application claims benefits and priority under 35 U.S.C. § 119(e) of the prior U.S. Provisional Patent Application No. 63/100,461 filed Mar. 13, 2020, which is incorporated in its entirety by reference herein.

### FIELD OF THE INVENTION

This invention relates to antiviral and antibacterial agents that are safe, nontoxic, noncorrosive and nonirritating to patients. The present invention can be employed as an antibacterial and an antiviral (encompassing cancer treatment or chemotherapy) for the treatment and prevention of infectious diseases. In a particular aspect, the present invention relates to compositions and methods for the treatment and prevention of viral (encompassing cancer treatment or chemotherapy) and bacterial infectious diseases by administering whole blood or plasma transfusion acquired from healthy children where it is permissible by law (emphasis added) or from healthy adults ages 18 to 20 years using hormones secreted by the thymus gland and their synthetic counterparts, a combination of them, their active compounds, their progenitors, and their stimulating and mediated compounds and their combinations thereof in hosts afflicted with viral and bacterial infections through thymopoiesis (the process of changing thymocytes to produce mature T cells) in an effort to produce mature T cells to fight such infections. The present compounds can also be employed as a preventive measure against these infections. The thymus gland produces 4 hormones; namely, thymosin, thymopoietin, thymulin and thymic humoral factor all of which play vital roles in both the immune and endocrine systems.

In another broad aspect, the present invention relates to compositions comprising thymus hormones that can be administered in a pharmaceutically acceptable vehicle or carrier other than healthy human blood or healthy plasma.

It is important to stress the fact here that the terms viral and antiviral include cancer since viral infections can cause cancer.

According to the instant invention, whole blood or plasma obtained from healthy children or from healthy adults ages 18 to 20 years administered to adult patients suffering from bacterial and viral infections such as COVID-19 can treat these infections. The compounds of the instant invention can also be administered to those individuals who are in need of such therapy such as those who have thymus diseases or those who are old enough that the thymus gland does not function anymore (thymus involution). Additionally, the present compounds can also be employed a priori to prevent such diseases by administering it to healthy individuals whereby antibodies are produced in the case of these subjects are exposed to such infections.

The thymus gland hormones that can be used by the instant invention are thymosin, thymopoietin, thymulin and thymic humoral factor. Synthetic thymus gland hormones can be used as well.

Since blood donation from children is considered illegal and unethical in most countries, an alternative approach would be to use healthy blood and healthy plasma from adults ages 18 to 20 years. Of course, donors will be fully informed and screened to make sure that they are healthy, and written consents of donors and their legal guardians, whenever ad hoc, should be properly documented in accordance with all local and federal laws before any blood is donated. Such logistics and legalities are done on regular bases and are routinely performed all over the World.

It is to be understood that the preceding general discussion and the discussion which follows are considered explanatory and exemplary in nature, and are solely intended to give additional merits of the current invention, as claimed.

Applicant specifically incorporates the entire contents of all cited references in this disclosure. All publications, patents, and patent applications mentioned herein form an integral part of the instant patent application and are incorporated in their entirety by reference herein.

### BACKGROUND OF THE INVENTION

Infectious diseases whether viral (encompassing cancer), bacterial or others can be detrimental to human health and also have great ramifications on the economy. Many compounds have been developed to treat such infections, but with limited success, especially those of viral origins. A few vaccines have been designed to stop the spread of viral infections but with limited success. This is because viral infections are ever evolving and medicine cannot cope with their novelty and mutations.

During the past 2 decades, there has been a growing strand of viral infections from a corona-shape like viruses such as SARS-CoV, MERS-CoV, and recently SARS-CoV-2 which causes COVID-19 infection that can be considered the deadliest amongst them all. Severe acute respiratory syndrome coronavirus 2 is a newly recognized viral infection, which has spread rapidly from China to the whole world. The first identified cases of SARS-CoV-2 were in early December of 2019 in Wuhan, which is the capital city of Hubei (a province in China). The virus has been identified as a novel enveloped RNA betacoronavirus 2 given the title name severe acute respiratory syndrome coronavirus 2 or SARS-CoV-2, which shares phylogenetic similarities to SARS-CoV (Lu R, et al. The Lancet. 395; 565-574 (2020)). The term COVID-19 has been designated to patients who have developed clinical symptoms without apparent radiological manifestations (Lu R, et al. The Lancet. 395; 565-574 (2020)). The overall clinical characteristics of COVID-19 mimic those of SARS-CoV in that fever and cough are its major symptoms, but gastrointestinal symptoms are almost absent, suggesting a clear difference in viral tropism when compared with SARS-CoV, MERS-CoV and seasonal influenza (Guan W, et al. The Lancet. 382; 1708-1720 (2020)). Another difference between COVID-19 and other known corona viruses is the fact that its infection was detected before the onset of viral pneumonia. The clinical spectrum of COVID-19 ranges from mild to critical and in many instances death for certain age group, especially those over 65 years of age (COVID-19 hospitalization and death by age. Aug. 18, 2020. https://www.cdc.gov/coronavirus/2019-ncov/covid-data/investigations-discovery/hospitalization-death-by-age.html).

Deciphering the origin of any zoonotic diseases and/or pandemics is paramount and is the most important factor since it unravels the underlying epidemiology and treatments of such diseases. Equally important is the fact that knowing the origins of these diseases will help in finding if there were “spill-over” incidents where such diseases pass directly from animals to humans, and most importantly precluding their reoccurrences. Delineating the origins of viruses requires years of extensive research, provided that it can be done, especially with the SARS-CoV-2 virus since, adding to its complexity, it is also mired with sensitive and tumultuous political issues between China and several countries of the World.

The taunting question that has perplexed the scientific community since its discovery is where the SARS-CoV-2 virus has originated from (Lu R, et al. The Lancet 395: 565-574 (2020); Dyer O. BMJ. Published Online Feb. 11, 2021. https://doi.org/10.1136/bmj.n428)? Videlicet, did the virus emerge naturally from bats or it was genetically engineered and leaked from the Wuhan Institute of Virology in China?

Since the onset of COVID-19 in the late 2019, the World is still probing for answers regarding SARS-CoV-19 virus' origin with no definite resolution on the horizon. What makes answering this question more problematic is the lack of transparency from China, lack of information about the virus' evolutionary history, place and time and mechanism of first transmission to humans.

A novel in vivo experiment that will conclusively answer this edifice without setting foot in China and/or acquiring more information from China is proposed by the instant invention. This robust in vivo experiment will decipher the origin of the SARS-CoV-2 virus with minimum data, which once and for all will set the records straight, and it will always give the right outcome regardless of the results (Batarseh K I. BMJ. Published Online Feb. 24, 2021. bmj.com/content/372/bmj.n428/rr-0). In the realm of logic, the concept of the outcome being always true such as in the instant of the proposed in vivo experiment is referred to as tautological equivalence since it will always provide the right answer.

The astute reader will realize that the support of the present experiment ensues from the fact that beta-coronaviruses are well-tolerated by bats. In fact, these viruses do not cause illnesses or deaths in bats primarily due to the remarkable and unique symbiotic relationship between the coronaviruses and bats where the bats are protected by their arsenal of sophisticated and exceptional immune systems (Briggs H. Published Online Jul. 22, 2020. https://www.bbc.com/news/science-environment-53494566; Banerjee A, et al. Scientific Reports. 10: 7257 (2020); Gorbunova V, et al. Cell Metabolism. 32: 31-43 (2020)).

What supports the present in vivo experiment is the commonality of the receptor-binding domain (RBD) of SARS-CoV-2 binds human ACE2 with high affinity (Wan Y, et al. J Virol. 2020: 94(7)), and if genetic modifications have been implemented, then one of the several reverse genetic systems available for beta-coronaviruses would have been employed (Cui J, et al. Nat Rev Microbiol. 17: 181-192 (2019)).

Accordingly, the proposed in vivo experiment involves infecting a statistically significant number of different species of bats with SARS-CoV-2. If the bats develop severe symptoms, disease manifestations and/or die, then the virus cannot have been originated from bats since as mentioned above bats tolerate this virus very well, but have emerged from the lab in China and the opposite holds true.

It is well-documented that there is a plethora of similarities between SARS-CoV-2 to SARS-CoV-like coronaviruses found in bats (Wu F, et al. Nature. 579: 265-269 (2020)), and 99.98% of SARS-CoV-2 genetic sequence are identical amongst humans (Lu R, et al. The Lancet 395: 565-574 (2020)). One Chinese species of special interest is the Rhinolophus affinis bat since a coronavirus with 96% of genomic material common to SARS-CoV-2 was found in this species (Zhou P, et al. Nature. 579: 270-273 (2020)).

As of Mar. 11, 2020 the total number of global COVID-19 laboratory-confirmed cases was 118,322 with a mortality of 4292 where most in China (https://www.who.int/docs/default-source/coronaviruse/situation-reports/20200311-sitrep-51-covid-19.pdf?sfvrsn=1ba62e57_8). The rate of infection related to COVID-19 has prompted the World Health Organization (WHO) on Mar. 11, 2020 to declare it a global pandemic.

These staggering infection cases with this high rate of mortality have prompted the scientific community to find a vaccine or a cure for COVID-19. Laboratories all over the world have been working diligently and around the clock to stop the spread of this deadly virus. Accordingly, there is an immediate need to develop vaccines and drugs to combat bacterial and viral infections such as COVID-19.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and in the appended claims.

### SUMMARY OF THE INVENTION

In accordance with the instant invention, new and safe compositions and methods are provided for the treatment and prevention of infections resulting from bacteria and viruses such as the family of corona viruses. The main impetus of the present invention stems from the fact that the nature of COVID-19 infectivity is that it is uncommon in children and children are insusceptible to this virus.

In accordance with the instant invention, it relates to the use of whole blood or plasma transfusion obtained from healthy children where it is permissible by law (emphasis added) or from healthy adults ages 18 to 20 years using hormones secreted by the thymus gland or their synthetic ones, a combination of them, their active compounds, their progenitors, and their stimulating and mediated compounds and their combinations thereof in hosts afflicted with viral and bacterial infections through thymopoiesis in an effort to produce mature T cells to fight such infections to treat patients suffering from bacterial and viral infections (encompassing cancer or chemotherapy) such as COVID-19 (Batarseh K I. BMJ. Published Online Jul. 13, 2020. bmj.com/content/368/bmj.m1252/rr-29; Batarseh K I. BMJ. Published Online Aug. 13, 2020. bmj.com/content/370/bmj.m2722/rr-4; Batarseh K I. BMJ. Published Online Dec. 1, 2020. bmj.com/content/371/bmj.m4670/rr-0; Batarseh K I. BMJ. Published Online Jan. 11, 2021. bmj.com/content/371/bmj.m4918/rr).

The compounds of the instant invention can also be administered to those individuals who are in need of such therapy such as those who have thymus diseases or those who are old enough that the thymus gland does not function anymore (thymus involution). Additionally, the present compounds can also be employed a priori to prevent such diseases by administering it to healthy individuals whereby antibodies are produced in the case of these subjects are exposed to such infections.

The thymus gland hormones that can be employed by the instant invention are thymosin, thymopoietin, thymulin and thymic humoral factor. Synthetic thymus gland hormones can be used as well.

Whole blood or plasma transfusion is already known to those skilled in the art and is being routinely performed in hospitals around the World. It is anticipated that whole blood or plasma transfusion acquired from healthy children where it is permissible by law (emphasis added) or from healthy adults ages 18 to 20 years will result in treating or preventing the diseases caused by such viruses or bacteria. The compositions of the instant invention can be administered in a pharmaceutically acceptable vehicle or carrier other than heathy human blood or healthy plasma.

It is noteworthy to mention here that the term viral encompasses cancer as well since there is an unequivocal link between viral infections and cancer where viruses affect the genomic make-up of healthy cells thereby turning healthy cells into cancerous ones. Some examples include, but not limited to, the human papillomavirus (HPV) that can cause cervical cancer and Epstein-Barr virus (EBV) that leads to Burkitt lymphoma and gastric adenocarcinoma (White M K, et al. Clin Microbiol Rev. 27; 463-481 (2014)).

The instant compounds can be used to treat and prevent viral infections such as the influenza viruses, the family of corona viruses, COVID-19, HIV, AIDS and the like and can be used as chemotherapeutic agents since viral infections can cause cancer.

The instant invention can also be used for the treatment and prevention of chickenpox, herpes, HPV, EBV, infectious mononucleosis, mumps, measles, rubella, shingles, gastroenteritis, hepatitis, meningitis, pneumonia, Ebola, septic shock, diabetes, arthritis, asthma, cirrhosis, allograft rejection, encephalomyelitis, vasculitis, lymphocytic choriomeningitis, glomerulonephritis, cancer, cachexia, myocarditis, an autoimmune disorder, psoriasis, urticaria, systemic lupus erythematosis, neuroblastoma, hematologic cancer, liver diseases, bacterial infections or psychoneuroimmunology of autoimmune disorders.

The term “chemotherapeutic” as used herein can be considered as the inhibition of the growth of cancer cells that are sensitive to the compositions disclosed by the instant invention. Preferably, such treatment can also lead to some regression of cancerous cells. Most preferably, such treatment leads to the near complete or complete regression of cancerous cells.

It is to be understood that both the foregoing general description and the following detailed description are only exemplary in nature and are not restrictive of the present invention, as claimed. All patents, patent applications, and publications mentioned throughout the present application are incorporated in their entirety by reference herein.

Additional features and advantages of the present invention will be set forth in part in the description that follows, and in part will be apparent from the description, or may be learned by practice of the instant invention. The objectives and other advantages of the present invention will be realized and attained by means of the elements and combinations particularly pointed out in the description and appended claims.

### DETAILED DESCRIPTION OF THE INVENTION

Passive immunization (PI) where antibodies or lymphocytes obtained from whole blood or plasma, which are produced by other individuals' immune systems, has long been used extensively for the prevention and treatment of infectious diseases (Keller M A, Stiehm E R. Clin Microbiol Rev. 13; 602-624 (2000)). The underlying concept of PI is to obtain antibodies or lymphocytes by collecting whole blood or plasma from patients who had survived a previous infection and acquired humoral immunity against that specific infection, and administering them to infected patients in an effort to eradicate such infections. There are different types of PI, including convalescent whole blood (CWB), convalescent plasma (CP) or convalescent serum (CS), monoclonal or polyclonal antibodies, pooled human immunoglobulin (Ig) for intravenous or intramuscular administration and high-titer human Ig.

Recently, CP was used to treat patients suffering from COVID-19 with severe symptoms (Duan K, et al. PNAS. 117; 9490-9496 (2020)). It was found that CP therapy was somewhat promising and it was well-tolerated by patients, leading to the disappearance of viremia, and improving symptoms and the clinical outcome of the disease.

Other drugs were evaluated such as hydroxychloroquine, lopinavir and ritonavir (Kaletra), interferon β 1a (SNG001), remdesivir tocilizumab (Actemra), and favipiravir (Avigan), which all proved ineffective against COVID-19 (Mahase E. BMJ. 368; M1252 (2020)). In addition, the anti-parasitic drug ivermectin was not found to be effective for the prevention or treatment of COVID-19, but reductions in viral load (IgG titers), cough and self-reported anosmia/hyposmia were observed (Chaccour C, et al. E Clinical Medicine. 100720 (2021)).

Very recent randomized double-blind clinical trials on fenofibrate, EXO-CD24 and Allocetra™ showed that these drugs were effective in the treatment of severely and critically COVID-19 patients. Data revealed that oral administration of fenofibrate was very promising in preventing lung damage in patients infected with COVID-19 (EurekAlert! AAAS. Promising clinical data for fenofibrate's ability to prevent lung damage in COVID patients. Published Online Dec. 22, 2020. https://www.eurekalert.org/pub_releases/2020-12/thuo-pcd122220.php). On the other hand, Exosomes-based drug EXO-CD24 inhalation stopped the cytokines storms that are typically associated with COVID-19 infectivity while the IV injection of Allocetra™ has shown significant recovery on COVID-19 patients and 14 out of 16 total patients were discharged from the hospital 5.3 days post treatment (Leichman A K. Has Israel just found the cure for Covid? Published Online on Feb. 7, 2021. https://www.israel21c.org/has-israel-just-found-the-cure-for-covid/).

The underlying principle of the instant invention is to use whole blood or plasma acquired from healthy children where it is permissible by law (emphasis added) or from healthy adults ages 18 to 20 years using hormones secreted by the thymus gland and their synthetic counterparts, a combination of them, their active compounds, their progenitors, and their stimulating and mediated compounds and their combinations thereof in hosts afflicted with viral and bacterial infections through the process of thymopoiesis in an effort to produce mature T cells to fight such infections to treat and prevent infections rather than the administration of vaccines, PI or other drugs such as the ones discussed above.

The thymus gland hormones that can be used by the instant invention are thymosin, thymopoietin, thymulin and thymic humoral factor. Synthetic thymus gland hormones can be used as well.

The rationale of the instant invention stems from the fact that COVID-19's infectivity is uncommon in children and children are insusceptible to it. Pediatric studies have shown that children are less susceptible to COVID-19 and death cases are extremely rare (Rasmussen S, Thompson L. JAMA Pediatr. 174; 743-744 (2020)). Only 2% of COVID-19 patients are younger than 19 years of age (Guan W, et al. NEJM. 382; 1708-1720 (2020)). Clinically, mild symptoms were observed with children without pneumonia and recovery was fast (Xu Y, et al. Nature Med 26; 502-505 (2020)).

The instant invention's approach is totally different than PI and particularly CP (Marano G, et al. Blood Trans. 14; 152-157 (2016)) since it employs healthy whole blood or healthy plasma obtained from healthy children or adults ages 18 to 20 years rather than previously infected whole blood or plasma obtained from patients who have overcome such infections. For the instant invention, the infected patient will produce the necessary antibodies to fight the infection through the administration of healthy whole blood or healthy plasma, contrary to PI where the antibodies are administered to the patient. The compounds of the instant invention can also be administered to those individuals who are in need of such therapy such as those who have thymus diseases, those who are old enough that the thymus gland does not function anymore (thymus involution) or subjects where thymectomy was performed. Additionally, the present compounds can also be employed a priori to prevent such diseases by administering it to healthy individuals whereby antibodies are produced in the case of these subjects are exposed to such infections.

One very crucial point merits special mention here, viz., once antibodies are produced as a result of prevention or treatment use of the compounds of the instant invention, these antibodies can be collected and administered to other infected patients in a similar way to that, for example, of CP therapy.

It is noteworthy to mention here that with respect to PT, there is always the case of insufficient recovered patients and consequently insufficient antibodies to be collected through apheresis to be administered for the treatment of sick individuals suffering from viral and bacterial infections, which makes PI therapy impractical. For COVID-19's patients for instance, CP is not feasible since only 1% of recovered patients have enough levels of antibodies to possibly neutralize the virus and their levels declined quickly, which was the case for the majority of patients tested for the presence of COVID-19's antibodies (NIH. Potent antibodies found in people recovered from COVID-19. Jun. 30, 2020. https://www.nih.gov/news-events/nih-research-matters/potent-antibodies-found-people-recovered-covid-19; REUTERS. Antibody levels in recovered COVID-19 patients decline quickly: research. Jun. 22, 2020. https://www.reuters.com/article/us-health-coronavirus-antibody/antibody-levels-in-recovered-covid-19-patients-decline-quickly-research-idUSKBN23T1CJ).

The insusceptibility of children to SARS-CoV-2 might be related to the fact that the thymus gland and its hormones remain active during puberty where the thymus grows and reaches its maximum size and then starts to atrophy and decay (thymus involution). Since the thymus is the primary site of de novo naïve T cell production, the presence of thymic hormones in the blood pool might be the reason for the resistance of children to COVID-19.

What further supports the notion of the thymus gland role is the fact that the concentration of thymopoietin was found to be greater than 1.0 ng/ml in normal plasma obtained from healthy infants and young adults while it was 0.25 ng/ml for healthy adults over 50 years of age (Twomey J J, et al. PNAS. 74; 2541-2545 (1977)). Additionally, data revealed that thymulin titers started to fall with increasing age where the average ranging from 4.77 log₂ for children 6.81 years of age to 0.55 log₂ for adults 58.1 years of age (Consolini R, et al. Clin Exp Immunol. 121; PMC1905732 (2000)).

According to the instant invention, whole blood or plasma acquired from healthy children where it is permissible by law (emphasis added) or from healthy adults ages 18 to 20 years enhances human thymopoiesis and prevents healthy subject and treats patients suffering from bacterial and viral infections such as COVID-19.

Of course, apheresis is also implemented in the instant invention since the therapeutic endogenous antibodies that are produced as a consequence of the implementation of the instant invention can still be administered to infected patients.

Previously, it was found that growth hormone (GH) therapy can in fact enhance thymopoiesis, thus increasing thymic hormonal output and the production of de novo naïve and total CD4+ T cells, which in turn facilitated immune restoration in HIV-1 patients (Napolitano L, et al. J Clin Invest. 118; 1085-1098 (2008)). The study concluded that thymic involution can be pharmacologically reversed and thymic function can be restored in immuno-deficient patients (Tesselar K, Miedema F. J Clin Invest. 118; 844-847 (2008); Napolitano L, et al. J Clin Invest. 118; 1085-1098 (2008)).

The thymus gland produces 4 hormones; namely, thymosin, thymopoietin, thymulin and thymic humoral factor all of which play vital roles in both the immune and endocrine systems. Any of these hormones, their progenitors, or chemical compounds where these hormones activate can be responsible for the resistance of children to COVID-19. One key essential hormone against COVID-19 might be the thymic humoral factor since it increases the immune system response to viral infections. Thus any of these hormones or their synthetic counterparts, a combination of them, their active compounds, their progenitors, and their stimulating and mediated compounds and their combinations thereof can be used in the instant invention.

Transfusion of heathy whole blood or healthy plasma is routinely performed and is a well-known technique to those skilled in the art.

The human immune system is comprised of two compartments: the myeloid and the lymphoid progenitors where both play essential and vital roles against all types of infections (Janeway, C A Jr, et al. The Components of the Immune System: In The Immune System in Health and Disease; 5^{th} Edition, Garland Science, New York, N.Y. (2001)). The myeloid progenitor produces granulocytes, dendritic cells, macrophages and mast cells. On the other hand, the lymphoid progenitor produces lymphocytic B and T cells where both originate from the bone marrow. The B cells (bone marrow derived) and T cells (thymus derived) mature in the bone marrow and the thymus, respectively. The B lymphocytes upon activation differentiate into plasma cells that secrete antibodies while the T cells encompass 2 classes of cells where one differentiate into cytotoxic T cells killing cells infected with viruses while the other class differentiate and activate other cells such as B cells.

As noted above, recovered COVID-19's patients do not produce robust antibodies that can manifest themselves for a prolonged period of time, making it difficult to produce an effective vaccine (REUTERS. Antibody levels in recovered COVID-19 patients decline quickly: research. Jun. 22, 2020. https://www.reuters.com/article/us-health-coronavirus-antibody/antibody-levels-in-recovered-covid-19-patients-decline-quickly-research-idUSKBN23T1CJ; Long Q, et al. Nature Med. 26; 1200-1204 (2020)). The immunity developed from COVID-19 infection is suboptimal, dysfunctional and short-lived ((REUTERS. Antibody levels in recovered COVID-19 patients decline quickly: research. Jun. 22, 2020. https://www.reuters.com/article/us-health-coronavirus-antibody/antibody-levels-in-recovered-covid-19-patients-decline-quickly-research-idUSKBN23T1CJ; Long Q, et al. Nature Med. 26; 1200-1204 (2020)). This surprising observation might be explained by the finding that only the myeloid response is hyperactivated while the lymphatic response is silenced (Haseltine W. The way that COVID-19 tricks the immune system could result in more severe illness. Forbes 2020. https://www.forbes.com/sites/williamhaseltine/2020/05/27/a-nasty-trick-in-the-covid-repertoire/?sh=3b9a689769e6). In essence, myeloid cell-mediated attacks the virus directly and causes significant inflammatory body response, creating a cytokine storm(s) and resulting in higher morbidity for elderly and severely diseased patients.

It appears that SARS-CoV-2 virus somehow is engineered to attenuate the lymphatic response and concurrently stimulate the myeloid response, which might explain the cytokine storm(s) and at the same time the absence or low levels of antibodies for moderately and severely diseased patients. This also might lend support to the fact that elderly patients are much more susceptible to SARS-CoV-2 than younger ones since T cells production ceases with increasing age, T cell senescence.

Transient lymphopenia is a common feature in most respiratory viral infections which occurs 2 to 4 days around the onset of symptoms and rapidly subsides (McClain M T, et al. J Clin Virol. 58; 689-695 (2013)); however, this is not true with COVID-19. Lymphopenia associated with COVID-19 is more pronounced and is prolonged, especially with severely diseased patients, and seems to be more selective towards T cell lineages (Chen G, et al. J Clin Invest. 130; 2620-2629 (2019)), emphasis added. In the case of COVID-19, lymphopenia is rarely observed in infected children where the mortality rate is almost zero, while in the elderly where the mortality rate is high, lymphopenia occurs more frequently with moderately and severely diseased cases, and always associated with significant decrease in T cell counts (Tavakolpour S, et al. Immunol Lett. 225; 31-32 (2020)).

Stress has also a profound effect on T cell functions and atrophy of the thymus. This as a result will negatively affect the homeostasis of the immune system, the production of T cells and therefore the lymphatic response to infections. Indeed, this was observed with COVID-19's infectivity (Debnath M, et al. Brain Behavior & Immunity. 100096 (2020)).

Collectively, it can be concluded that T cells impairment and downregulation are the culprit and might be the major player in the treatment of bacterial and viral infection such as COVID-19. It is well documented that T cells that survive the selection process in the thymus differentiate into CD4+ or CD8+ single positive T cells, which are essential in protective immunity and the production of antibodies. Studies have shown that both CD4+ T and CD8+ T memory cells were found in 100% and 70% of COVID-19's recovered patients, respectively (Grifoni A, et al. Cell. 181; 1489-1501 (2020)).

The mounting evidence presented here supports the supposition suggested earlier regarding the notion that administration of whole blood or plasma obtained from healthy children or healthy young people will enhance human thymopoiesis and will treat patients suffering from bacterial and viral infections such as COVID-19, producing T cells and their differentiating counterparts CD4+ and CD8+ cells (Batarseh K I. BMJ. Published Online Jul. 13, 2020. bmj.com/content/368/bmj.m1252/rr-29; Batarseh K I. BMJ. Published Online Aug. 13, 2020. bmj.com/content/370/bmj.m2722/rr-4). As a side note, it is also anticipated that this method of treatment might be able to alleviate stress-mediated inflammatory response to COVID-19, viz., psychoneuroimmunology (Ader R. Psychoneuroimmunology. New York, N.Y.: Academic Press (1981b)).

Accordingly, the treatment scheme presented by the instant invention through the administration of whole blood or plasma obtained from healthy children where it is permissible by law (emphasis added) or from healthy adults ages 18 to 20 years will excite and fine-tune the human immune system via the process of thymopoiesis, and thereby treating such infections, and endogenously produce antibodies that can neutralize bacteria and viruses. As mentioned earlier, almost all moderately and severely COVID-19's patients are age advanced, which is associated with impaired T cells and downregulation of both CD4+ T and CD8+ T cells responses.

The thymus is capable of regenerating and restoring its endogenous functions to a certain degree, but it is unable to fully restore its functionality in the aged population, which requires the need for exogenous therapies to aid in its regeneration (Chaudhry M S, et al. Immunol Rev. 271; 56-71 (2016)). Some of these exogenous therapies include interleukin IL-7 (Phillips J A, et al. J Immunol. 173; 4867-4874 (2004)), keratinocyte growth factor (KGF) also known as fibroblast growth factor 7 (Finch P W, Rubin J S. Adv Cancer Res. 91; 69-136 (2004)), hormonal modulation such as growth hormones (GH) and thymus bioengineering (Chung B, et al. Stem Cells. 32; 2386-2396 (2014)).

Indeed, it was found previously that GH therapy can in fact enhance thymopoiesis, increasing thymic hormonal output and the production of de novo naïve and total CD4+ T cells, which in turn facilitated immune restoration in HIV-1 patients (Napolitano L, et a. J Clin Invest. 118; 1085-1098 (2008)). The study concluded that thymic involution can be pharmacologically reversed and thymic function can be restored in immuno-deficient patients (Napolitano L, et al. J Clin Invest. 118; 1085-1098 (2008)).

Thus and according to the present invention, whole blood or plasma procured from healthy children where it is permissible by law (emphasis added) or from healthy adults ages 18 to 20 years can enhance human thymopoiesis and treat patients suffering from bacterial and viral infections such as COVID-19 by the endogenous production of antibodies through the exogenous administration of whole blood or plasma from healthy individuals via thymopoiesis. The mounting evidence presented here supports the notion that administration of whole blood or plasma obtained from healthy children where it is permissible by law (emphasis added) or from healthy adults ages 18 to 20 years will promote human thymopoiesis and treat patients suffering from bacterial and viral infections such as COVID-19, producing T cells and their differentiating counterparts CD4+ and CD8+ cells (Batarseh K I. BMJ. Published Online Jul. 13, 2020. bmj.com/content/368/bmj.m1252/rr-29; Batarseh K I. BMJ. Published Online Aug. 13, 2020. bmj.com/content/370/bmj.m2722/rr-4; Batarseh K I. BMJ. Published Online Dec. 1, 2020. bmj.com/content/371/bmj.m4670/rr-0; Batarseh K I. BMJ. Published Online Jan. 11, 2021. bmj.com/content/371/bmj.m4918/rr).

Vaccination plays an extremely vital role in the fight against bacterial and viral infections (cancer as well) and is an essential and robust component in infectious diseases' armamentarium. Just couple of months ago, three of the most leading pharmaceutical companies AstraZeneca/University of Oxford, Pfizer Inc. with its German-based partner BioNTech and Moderna have announced to the World the anticipated and great news regarding the mass production of 3 vaccines that showed very promising clinical results against COVID-19. The AstraZeneca/University of Oxford ChAdOx1 nCoV-19 vaccine resulted in a 62% to 90% efficacy, depending on dosing (Mahase E. BMJ. Published Online Nov. 27, 2020. https://www.bmj.com/content/371/bmj.m4670) while the Pfizer/BioNTech BNT162b2 mRNA Covid-19 vaccine gave 95% efficacy (Polack F, et al. NEJM. 383; 2603-2615 (2020)), and finally the Moderna mRNA-1273 SARS-CoV-2 vaccine gave 94.1% efficacy (Baden L R, et al. Published Online Dec. 30, 2020. doi: 10.1056/NEJMoa2035389). The three vaccines differ in their design and mode of action in that the AstraZeneca/University of Oxford vaccine employs viral vector-based vaccine while the other two use messenger RNA or mRNA to combat COVID-19.

Regardless of the differences in technology and underlying design, these vaccines have proven effective in preventing COVID-19 infection as determined by clinical trials as mentioned above. However, amongst the obvious questions and problems that need further investigation in the future development of robust COVID-19 vaccines, there are 5 major unprecedented caveats with the present ones; namely, a) COVID-19 is unlike any other flu infection since it is not a seasonal flu, but it is a yearly round flu and infections can occur at any time, b) it is crucial to determine how long the immunity lasts after prime vaccination since this might require many additional or boost vaccination in a short period of time, c) it is also of concern to examine how COVID-19 vaccines interact with the other known seasonal flu vaccines, d) another important aspect that requires a thorough investigation is that what will guarantee that these vaccines in certain individuals and on the long run will not activate the myeloid progenitor thereby creating deadly cytokine storms instead of the therapeutic lymphocytic one, and finally e) it is possible that healthy cells and the immune system will be genetically altered, especially with those vaccines that employ mRNA technology, which might adversely affect the genetic make-up of healthy cells.

A closer inspection of these 5 caveats, one cannot escape the fact that doses of vaccine should be administered continuously, which might develop resistance on the long run. Equally important is the fact that there might be a contraindication and detrimental health effects of COVID-19's vaccines once administered with other seasonal flu vaccines. It is also possible that vaccination might activate the harmful myeloid system, which is responsible for the production of deadly cytokines storms instead of the therapeutic lymphocytic system. Furthermore, these vaccines that are based on mRNA technology might, on the long run, affect the genetic make-up of healthy cells and their signaling, resulting in the development of newly and genetically altered diseases that no one can predict their outcomes.

These unknowns need to be addressed very carefully and diligently since these caveats might compromise our immune system. The taunting question here is how would a healthy immune system respond to these multiple vaccinations especially when it is bombarded by these foreign bodies at all times? Could this result in the mutation of a corona virus that is even worse than SARS-CoV-2! What makes this even more worrying is the fact that it was previously found that recovered COVID-19 patients do not produce robust antibodies that can manifest themselves for a lengthy period of time, making it difficult to produce an effective vaccine (Batarseh K I. BMJ. Published Online Jul. 13, 2020. bmj.com/content/368/bmj.m1252/rr-29; Batarseh K I. BMJ. Published Online Aug. 13, 2020. bmj.com/content/370/bmj.m2722/rr-4). The immunity developed from COVID-19 infection is suboptimal, dysfunctional and short-lived (Batarseh K I. BMJ. Published Online Jul. 13, 2020. bmj.com/content/368/bmj.m1252/rr-29; Batarseh K I. BMJ. Published Online Aug. 13, 2020. bmj.com/content/370/bmj.m2722/rr-4). This surprising observation might be explained by the finding that only the myeloid response is hyperactivated while the lymphatic response is silenced in COVID-19 infection (Haseltine W. The way that COVID-19 tricks the immune system could result in more severe illness. Forbes 2020. https://www.forbes.com/sites/williamhaseltine/2020/05/27/a-nasty-trick-in-the-covid-repertoire/?sh=3b9a689769e6).

In essence, myeloid cell-mediated attacks the virus directly and causes significant inflammatory body response, creating a cytokine storm(s) and resulting in higher morbidity for the elderly and severely diseased patients, including patients with comorbidities. It is documented that some vaccines can actually activate the myeloid immune system, and results have shown that post-boost responses have given rise to markers involving costimulation, chemotaxis, inflammation and phagocytosis (Palgen, J L, et al. Sci Rep. 8; 3087 (2018)). Since it is hopeful that COVID-19's vaccines will produce enough therapeutic antibodies-provided that they will do-to neutralize COVID-19 infection, it appears that such immunity might not be present in all individuals and is short lived.

The fast-track efficacy clinical studies of Pfizer/BioNTech's vaccine BNT162b2 mRNA Covid-19 was based on double-blind Randomized Control Trials (Polack F, et al. NEJM. 383; 2603-2615 (2020)). By carefully scrutinizing the clinical trial's protocols and design adopted by Pfizer/BioNTech, it can be concluded that no plasma COVID-19 antibodies were detected and measured, which is key for determining efficacy. The serological detection and measurement of antibodies for COVID-19 infection is tantamount since 40% of infected people are asymptomatic (COVID-19 Pandemic Planning Scenarios. CDC. Updated Sep. 10, 2020. https://www.cdc.gov/coronavirus/2019-ncov/hcp/planning-scenarios.html), and recovered COVID-19 patients do not produce enough robust antibodies that can manifest themselves for a lengthy period of time (Batarseh K I. BMJ. Published Online Jul. 13, 2020. bmj.com/content/368/bmj.m1252/rr-29; Batarseh K I. BMJ. Published Online Aug. 13, 2020. bmj.com/content/370/bmj.m2722/rr-4). The immunity developed from COVID-19 infection is suboptimal, dysfunctional and short-lived (Batarseh K I. BMJ. Published Online Jul. 13, 2020. bmj.com/content/368/bmj.m1252/rr-29; Batarseh K I. BMJ. Published Online Aug. 13, 2020. bmj.com/content/370/bmj.m2722/r-4). Collectively, this will skew the efficacy results and affect the prevalence of COVID-19 cases in both groups.

Data obtained from well-controlled immunogenicity studies suggested that vaccinated subjects with BNT162b2 mRNA Covid-19 Pfizer/BioNTech's vaccine showed strong dose-dependence antibodies response for SARS-CoV-2, and broad protection against 19 diverse mutants (Mulligan M J, et al. Nature. 586; 589-593 (2020); Sahin U, et al. Nature. 586; 594-599 (2020)). Results confirmed the induction of functional and pro-inflammatory CD4+ and CD8+ T cell responses in almost all trial subjects with TH1 polarization with the concurrent production of modulatory immune cytokines IFNγ. These vaccine-induced antibodies were compared with those procured from confirmed COVID-19 patients (convalescent) and in vitro comparison was more than satisfactory. However, the effectiveness of these antibodies was not tested. A more robust and accurate study is to test the efficacy of these antibodies by administering them to moderately and severely infected COVID-19 patients, mimicking protocols used for convalescent plasma. Finally, based on COVID-19's clinical infection end points, the efficacy can then be assessed and calculated.

Another issue that should have been considered with the Pfizer/BioNTech vaccine is whether the participants maintained social distancing throughout the duration of the clinical trial since maintaining social distancing can significantly affect the outcome. By examining the data of Table 3 (Polack F, et al. NEJM. 383; 2603-2615 (2020)), it can be concluded that for both groups older participants have less frequency of infection, which might be attributed to older participants are usually more careful and vigilant to follow social distancing than younger subjects! Incidentally, the authors did not discuss this observation, and no information regarding social distancing was provided in the efficacy clinical trial (Polack F, et al. NEJM. 383; 2603-2615 (2020)).

It is also noteworthy to mention here that blood type O patients have a lower risk of COVID-19's infection and a reduced likelihood of severe symptoms and even death (Zietz M, et al. Nature Commun. 11; Article No. 5761; 1-6 (2020)). Therefore, determination of ABO blood type and phenotypes should have been performed on those participants who developed COVID-19 infection for the clinical trial of the Pfizer/BioNTech vaccine.

For the Pfizer/BioNTech vaccine, it was found that only 1 severe case in the vaccinated group was observed out of 8 total confirmed cases while 9 severe cases were observed for the placebo group out of 162 confirmed cases. My calculations revealed that the percentages of severe cases of COVID-19 infection for the vaccinated and the placebo groups are 12.5% ([1/8]×100%) and 5.55% ([9/162]×100%), respectively. This means, on the average, that more severe cases of COVID-19 are found in the vaccinated group than the placebo group, which is very disturbing.

I have also calculated the chi-square χ² value obtained by applying the Mantel-Cox or logrank statistical test for the above severe cases for both groups and comparing it with the tabulated one at 95% confidence interval and 1 degrees of freedom (Bland J M, Altman D G. BMJ. 328; 1073 (2004)). The results have shown that the difference is highly significant, lending further support that severe cases in the vaccinated group are statistically more prevalent than that for the placebo, confirming the above average calculations.

The Moderna mRNA-1273 SARS-CoV-2 vaccine was granted emergency use authorization (EUA) by the United States Food & Drug administration (USFDA) as an effective immunization against COVID-19 on Dec. 18, 2020 (Tanne J H. Published Online Dec. 21, 2020. BMJ 2020; 371:m4924), and on Jan. 8, 2021 by the Medicines and Healthcare Products Regulatory Agency (MHRA) of the United Kingdom (Mahase E. Covid-19. Published Online Jan. 11, 2021. BMJ 2021; 372:n74). The % efficacy for the Moderna vaccine ages 18 to <65 years was calculated to be 95.6% while for ages ≥65 years was 86.4% (Fact sheet for healthcare providers administering vaccine (vaccination providers) emergency use authorization (EUA) of the Moderna COVID-19 vaccine to prevent coronavirus disease 2019 (COVID-19). https://www.fda.gov/media/144637/download. Last date accessed Jan. 14, 2020; Baden L R, et al. Published Online Dec. 30, 2020. doi: 10.1056/NEJMoa2035389).

During the EUA discussion, an issue was raised by the USFDA's panel regarding the above differences in efficacy values as a function of age. Moderna has attributed this incongruous observation to the fact that few COVID-19 cases were included in the age group ≥65 years (Branswell H. A side-by-side comparison of the Pfizer/BioNTech and Moderna vaccines. Published Online Dec. 19, 2020. https://www.statnews.com/2020/12/19/a-side-by-side-comparison-of-the-pfizer-biontech-and-moderna-vaccines/). The rationale given by Moderna is moot for the mere fact that the % efficacy is not a whole number but a ratio which should be independent of the total number of participants. As the denominator is increased so does the numerator increase by the same amount and vice versa in order to keep the ratio constant; otherwise, the % efficacy will not be an absolute number anymore. If one uses Moderna's argument, then the % efficacy is a variant that changes as a function of the number of participants, which is incorrect. Yes, there can be slight variations in the calculation of the % efficacy, but not the large variation observed by the Moderna vaccine. Does this mean that if Moderna tests more participants, then the efficacy at some point will reach 100%, which is straightforward abhorrent? The above discrepancy in % efficacy can actually be explained scientifically.

The reason behind the above age-dependence discrepancy in the % efficacy might be directly related to the thymus gland and not the number of cases in the age group ≥65, contradicting what Moderna has suggested. As discussed previously, it is well-established and documented that the thymus gland undergoes a peculiar feature, viz, it degenerates and deteriorates as we age, albeit at a much faster pace than other organs, leading to the reduction of thymopoieses. It is also well-known that the thymus is the primary site of de novo naïve T cell production expressing αβ-type T cell antigen receptors (Batarseh K I. BMJ. Published Online Jul. 13, 2020. https://www.bmj.com/content/368/bmj.m1252/rr-29; Batarseh K I. BMJ. Published Online Aug. 13, 2020. https://www.bmj.com/content/370/bmj.m2722/rr-4). Thus, the thymus gland and its hormones remain active during puberty where the thymus grows and reaches its maximum size and then undergoes progressive atrophy, a process known as involution. This process of deterioration starts from birth and accelerates as we age, and by age of 65 (emphasis added) the thymus is basically has been impaired enough that it is unable to make new T cells, and thereby diminishing our adaptive immune response, leaving us prone to infections and making vaccines less effective (Steinmann G G, et al. Scand J Immunol. 22; 563-575 (1985); Taub D D, Longo D L. Immunol Rev. 205; 72-93 (2005); Naylor K, et al. J Immunol. 174; 7446-7452 (2005)).

Data obtained from epidemiological and immunological studies have shown that the dramatic increase in incidence of age-related infectious diseases and cancer is due to immunosenescence and ensuing inflammaging, which is directly correlated with decline in thymic T cell production (Thomas R, et al. Immun Ageing. 17; 1-17 (2020)). The decline of thymic T cell production T(t), which plays a significant role in immunosenescence, is exponentially related to time (t) and can be expressed by Equation 1 as (Palmer S, et al. PNAS. 115; 1883-1888 (2018)):

T(t)=e^{-α1} (1)

where α is a constant equaling 0.044 year⁻¹ with a half-life of approximately 15.7 years (Palmer S, et al. PNAS. 115; 1883-1888 (2018)). Further along these lines, it was shown that by age 55 only 5% of naïve T cell production results from thymic output (Murray J M, et al. Immunol Cell Biol. 81; 487-495 (2003)).

Collectively, it can be deduced from the above that the difference in the % efficacy observed with the Moderna vaccine cannot simply be related to the low number of participants in the age group of >65, but actually to thymic involution and the cessation of naïve T cell production. It is highly improbable that the cut-off age of % efficacy of 65 years can exactly coincide with the same time that the thymic output also completely diminishes as stated above. This remarkable observation cannot just be a mere coincidence, considering our scientific knowledge and what we already know about the thymus gland. Therefore, Moderna should look into thymic involution as a plausible explanation regarding the observed discrepancy of % efficacy, and not just offer the above naïve justification in order to dismiss these unorthodox results (Batarseh K I. BMJ. Published Online Feb. 15, 2021. https://www.bmj.com/content/372/bmj.n74/rr-7). Finally, what is also very perplexing is the fact that the USFDA did not even object on the mundane rationale given by Moderna regarding the discrepancy of % efficacy!

Like its predecessor the Pfizer/BioNTech vaccine, no information was provided regarding whether the participants maintained social distancing throughout the duration of the Moderna vaccine clinical trial (Batarseh K I. Published Online Jan. 11, 2021. https://www.bmj.com/content/371/bmj.m4918/rr). This is important because social distancing will favorably skew the calculated % efficacy. The USFDA and its advisory committee should have addressed this paramount issue before issuing the EUA. The significance of addressing this issue should not come from the public, but from the USFDA and its advisory committee. Nonetheless, if this issue was discussed by the appropriate panel, then this information should have been incorporated in the EUA and should have readily been available to the public.

All three vaccines discussed above have variable side-effects ranging from mild to severe, including anaphylactic shocks, and can even result in deaths (Ramasamy M N, et al. Lancet. 396; 1979-1993 (2020); Polack F, et al. NEJM. 383; 2603-2615 (2020); Baden L R, et al. Published Online Dec. 30, 2020. doi: 10.1056/NEJMoa2035389). Mild symptoms include fever, fatigue headache, aching, chills, joint pain, and pain at the injection site. Most people have experienced these mild symptoms after the second dose than after the first one. It is extremely important to stress the fact that vaccination is a preventive and not a curative measure since individuals who have been vaccinated can still contract the virus. Therefore, one has to continue to be diligent and careful when vaccinated.

Despite the above caveats and shortcomings, no one can deny the fact that the fast-track design of these vaccines through Operation Warp Speed, which was initiated and announced and by President Donald Trump on May 15, 2020, can be considered a great scientific and medical miracle. The development of these well-designed vaccines was accomplished in less than a year from the emergence of COVID-19 from China, making it undoubtedly remarkable!

The above grim outlook might put these vaccines under stringent scrutiny for their safety and effectiveness. Therefore, there is an immediate need to develop safer methods or a cure rather than a vaccine to combat these deadly bacterial and viral infections such as COVID-19. Otherwise, the best solution might presently be to continue with the current mitigation measures and locked downs since such measures have proven to be extremely effective—with the concomitant tumultuous negative impact on the economy—in combating and substantially reducing the spread of COVID-19 infection (Batarseh K I. Published Online Jun. 14, 2020. Science. https://science.sciencemag.org/content/368/6489/395/tab-e-letters) or until the holy grail of vaccines is discovered. However, a more robust alternative is to find a cure (emphasis added) such as the method presented in the instant invention.

Accordingly, in an effort to develop safer and more effective methods for the treatment of infectious diseases, the present invention describes compounds that are safe, nontoxic, noncorrosive and nonirritating and methods thereof that are antibacterial and antiviral (encompassing cancer as well) for the treatment of infections in subjects suffering from such diseases via the process of thymopoiesis. The method relies on administering whole blood or plasma transfusion obtained from healthy children where it is permissible by law (emphasis added) or from healthy adults ages 18 to 20 years to infected patients. This exogenous method will result in the endogenous regeneration of the thymus gland to produce mature T cells, which are extremely important in adaptive immunity. The compounds of the instant invention can be those, but not limited to, hormones secreted by the thymus gland and their synthetic counterparts, a combination of them, their active compounds, their progenitors, and their stimulating and mediated compounds and their combinations thereof can be used in the instant invention.

The composition may further include DNA-intercalating agents and agents that inhibit the activity of topoisomerase I and topoisomerase II. The composition may further include cytokines, antimicrobial agents, antiviral agents, hormones, sedatives, hypnotics, tranquilizers, topically active drugs, and vasodilating substances.

According to one aspect of the present invention, several DNA-intercalating agents can be added as well such as those associated with the ability to inhibit the enzymes topoisomerase I and topoisomerase II which are responsible for the interconversion of the topological states during DNA transcription and replication, and the regulation of DNA supercoiling. Examples of topoisomerase I inhibitors include protoberberines alkaloids and their synthetic analogs, coralyne, the benzo[c]phenanthridine alkaloids and nitidine as well as the fungal metabolites, bulgarein, camptothecin and its derivatives topotecan and irinotecan, bi- and terbenzimidazoles, indolocarbazole derivatives, and saintopin. Other topoisomerase I inhibitors are beta-lapachone, diospyrin, topostatin, topostin, favonoids, Hoechst 33258 and the like and mixtures thereof. Examples of topoisomerase II inhibitors include teniposide or epipodophyllotoxin, VP-16 and VM-26, and podophyllotoxin-acridine conjugates-pACR6 and pACR8, the compositions describe in U.S. Pat. No. 8,048,870 and the likes and mixtures thereof.

In particular embodiments, the present compositions may be administered in combination with cytokine such as, but not limited to, interleukins; antimicrobial agents such as, but not limited to, acyclovir, chloramphenicol, chlortetracycline, itraconazole, mafenide, metronidazole, mupirocin, nitrofurazone, miconazole, magainins, cecropins, defensins, oxytetracycline, penicillin, tetracycline, and those compositions described in U.S. Pat. Nos. 6,242,009, 6,630,172 and 8,673,366, which are hereby incorporated in their entirety by reference.

Hormones such as, but not limited to, GH, adrenocorticosteroids, cortisone, cortisol, betamethasone benzoate, betamethasone valerate, desonide, fluocinolone acetonide, halcinonide, and hydrocortisone; sedatives, hypnotics and tranquilizers such as, but not limited to, metandienone, benzocaine, dibucaine, lidocaine, pramoxine hydrochloride and tetracacine, pentobarbital sodium, phenobarbital, secobarbital sodium, carbromal, sodium phenobarbital, reserpine, and thiopropazate hydrochloride; analgesics such as, but not limited to, camphor, and menthol; vasodilating substances such as tolazoline; thrombosis-hampering substances such as heparin; certain biological substances which affect tissue formation and tissue stabilization such as EGF (epidermal growth factor), EGF-URo (EGFurogastron), and somatotropin asellacrine can also be incorporated in the instant invention.

Other drugs that exhibits antibiotic and antitumor activities such as bleomycin, amsacrine, mitomycin C, adriamycin, actinomycin D, daunomycins, neocarsinostatin, steptonigrin, elliptinium acetate, doxorubicin, epirubicin, mitoxantrone, idarubicin, daunorubicin, mitomycin and the likes or mixtures thereof can all be used.

Also contemplated in the present invention is the inclusion of various antiviral agents. Examples of these agents include 9-(2-Hydroxyethoxymethyl) guanine, ZOVIRAX (GlaxoWelicome), idoxuridine, trifluorothymidine, bromovinyldeoxyuridine, ribavirin, amantadine, rimantadine, nevirapine (NVP), and the likes or mixtures thereof can all be used.

Certain embodiments of the present invention include angiogenesis inhibitory drugs such as angiostatin and endostatin, and the likes or mixtures thereof can all be used.

Other drugs which can be a part of the instant invention are fenofibrate, EXO-CD24, Allocetra™, hyrdroxychloroquine, remdesivir, ivermectin, tocilizumab, lopinavir, ritonavir, camostat, famotidine, sarilumab, nitazoxanide, corticosteroids, nafamostat, bevacizumab, fluvoxamine, umifenovir, and the likes or mixtures thereof can all be used.

The present compositions are safely given to patients, and therefore those of skill in the art recognize that the compositions described herein can be ingested or administered to patients via intramuscular (IM), intravenous (IV) intraperitoneal (IP) or intrathecal (IT) route of injection or any other effective route to the site of infection or tumor. The compositions further comprise pharmaceutically acceptable stabilizers, adjuvants, diluents, vitamins, minerals, zinc, dietary supplements, and mixtures thereof, and other components that are well known to those skilled in the art. The antibiotics, antiviral compounds, the chemotherapeutic agents, and the analgesics can be added singularly to the compositions of the present invention, or in combination with each other.

Other embodiments of the instant application include endogenous and exogenous thymus regeneration therapies, including stem cells and thymus bioengineering in the case of thymectomy (Chaudhry M S, et al. Immunol Rev. 271; 56-71 (2016); Shichkin V P, Antica M. Stem Cell Rev and Rep. 16; 239-250 (2020)).

The compositions of the instant invention may be conveniently administered in a pharmaceutically acceptable liquid carrier that is approved for such uses other than healthy human blood or healthy plasma. Any liquid carrier may be employed provided that such carrier should behave as an inert, that is, should not chemically interfere with the chemical constituents of the present compositions. These vehicles or carriers should be harmless, nontoxic, noncorrosive and nonirritant and should have pH values between 5.5 (saline solution average pH value) to 7.45 (pH range of healthy or normal human blood or plasma is between 7.35 to 7.45) where the average pH value of a healthy human blood or plasma is 7.4 (Kellum J A. Crit Care. 4; 6-14 (2000)).

Examples of such carriers are crystalloids, saline solutions and lactated Ringer's, also known as sodium lactate and Hartmann's solutions and the likes. Of course, there are contraindications for the use of these carriers in some patients, which are already known to those skilled in the art (Lippincott Nursing Center. IV Fluids. https://www.nursingcenter.com/getattachment/Clinical-Resources/nursing-pocket-cards/IV-Fluids/IV-Fluids-_January-2019.pdf.aspx (January, 2019)), and caution should be exercised when treating these patients. Other delivery routes will be readily apparent to those skilled in the art.

Applicant specifically incorporates the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof and all integers and fractions within the range. It is not intended that the scope of the invention be limited to the specific values recited when defining a range.

The amount of composition of the instant invention added depends on numerous factors, including its function, the time needed to accomplish this function, severity of the disease being treated, and the like. Therefore, it is prudent to know a priori information about the severity of the pathological disorder and host being treated in an effort to administer the proper concentration. Preferably, the amount of the composition added should be at least equal the concentration present in the whole blood or plasma of healthy children or from healthy adults ages 18 to 20 years. For example, the amount of thymosin should be at least 16.3 μg/ml (Hannapel E, van Kampen M. J Chromatography A. 397; 279-285 (1987)), for thymopoietin should be at least 1.0 ng/ml (Twomey J J, et al. PNAS. 74; 2541-2545 (1977)), for thymulin should be at least 4.77 log₂ (Consolini R, et al. Clin Exp Immunol. 121; PMC1905732 (2000)), and finally for thymic humoral factor should be at least 93 nmol/ml (Martignoni M, et al. Proteins & Proteomics. 1868; 140467 (2020)).

Since the activity of thymulin depends on the presence of zinc in the molecule, dietary zinc supplementation is required in case there is a plasma zinc deficiency in subjects in need of the instant invention (Prasad A S, et al. J Clin Invest. 82; 1202-1210 (1988)).

It will be appreciated that the actual preferred method and administration via intramuscular (IM), intravenous (IV) intraperitoneal (IP) or intrathecal (IT) route of injection will vary according to the particular pathological disorder. The optimal method and order of administration of the subject composition for a given set of conditions can be ascertained by those skilled in the art using conventional techniques and in view of the information set out herein by the present invention.

Other embodiments of the present invention will be apparent to those skilled in the art from consideration of the present specification and practice of the present invention disclosed herein. It is intended that the present specifications and examples be considered as exemplary only with a true scope and spirit of the invention being indicated by the instant claims and equivalents thereof.

The preceding discussion, examples, procedures, and specific salient features disclosed herein are solely intended for setting-up the general and certain preferred embodiments of the present invention. Thus, various modifications and suggestions in lieu thereof can be implemented by those skilled in the art. These should be an essential part of this application and can be made consistent with the letter and spirit of the foregoing disclosure, and within the true scope, merits and purview of this invention, which is presented by the following claims.

What is claimed is:

1. A method of treating infected subjects, the method comprising the step of administering to a subject in need thereof thymus gland hormones.

2. The method of claim 1, wherein the method can also be employed as a preventing measure in healthy subjects in case of exposure to infections.

3. The method of claim 1, wherein the method can also be employed in subjects where thymectomy was performed, including stem cells and thymus bioengineering.

4. The method of claim 1, wherein the infections are viral infections.

5. The method of claim 1, wherein the infections are bacterial infections.

6. The method of claim 1, wherein the infections are cancer.

7. The method of claim 1, wherein the thymus gland hormones enhance the endogenous regeneration of the thymus gland via the process of thymopoiesis, thereby promoting the production of mature T cells.

8. The method of claim 1, wherein the thymus gland hormones can be those, but not limited to, hormones secreted by the thymus gland, a combination of them, their active compounds, their progenitors, and their stimulating and mediated compounds and their mixtures thereof.

9. The method of claim 1, wherein the thymus gland hormones are thymosin, thymopoietin, thymulin and thymic humoral factor.

10. The method of claim 1, wherein the thymus gland hormone are found in healthy whole blood or healthy plasma transfusion procured from healthy children where it is permissible by law.

11. The method of claim 1, wherein the thymus gland hormones are found in healthy whole blood or healthy plasma transfusion procured from healthy adults ages 18 to 20 years.

12. The method of claim 1, wherein the pH of healthy children blood or plasma is between 7.35 to 7.45.

13. The method of claim 1, wherein the pH of healthy adults blood or plasma ages 18 to 20 years is between 7.35 to 7.45.

14. The method of claim 1, wherein the thymus gland hormones can be of synthetic nature.

15. The method of claim 1, wherein the human thymus gland hormone is administered in an effective amount equaling to at least to the concentration of the hormone present in children healthy whole blood or healthy plasma or adults healthy whole blood or healthy plasma ages 18 to 20 years.

16. The method of claim 1, wherein the human thymus gland hormone is thymosin administered in a concentration of at least 16.3 μg/ml.

17. The method of claim 1, wherein the human thymus gland hormone is thymopoietin administered in a concentration of at least 1.0 ng/ml.

18. The method of claim 1, wherein the human thymus gland hormone is thymulin administered in a concentration of at least 4.77 log₂.

19. The method of claim 1, wherein the human thymus gland hormone is thymic humoral factor in a concentration of at least 93 nmol/ml.

20. A composition of claim 1, comprising a pharmaceutically acceptable carrier.

21. The composition of claim 1, wherein the pharmaceutically acceptable carrier comprises crystalloids, saline solutions and lactated Ringer's.

22. The composition of claim 1, wherein the pharmaceutically acceptable carrier has a pH range values between 5.5 to 7.45.

23. The method of claim 1, wherein the composition can be administered to a subject via intramuscular (IM), intravenous (IV) intraperitoneal (IP) or intrathecal (IT).

24. The method of claim 1, wherein the infections include chickenpox, influenza, family of corona viruses, herpes, HIV, AIDS, human papillomavirus (HPV), Epstein-Ban virus (EBV), infectious mononucleosis, mumps, measles, rubella, shingles, gastroenteritis, hepatitis, meningitis, pneumonia, Ebola, septic shock, diabetes, arthritis, asthma, cirrhosis, allograft rejection, encephalomyelitis, vasculitis, lymphocytic choriomeningitis, glomerulonephritis, cancer, cachexia, myocarditis, an autoimmune disorder, psoriasis, urticaria, systemic lupus erythematosis, cancer, neuroblastoma, hematologic cancer, Burkitt lymphoma, gastric adenocarcinoma, liver diseases, bacterial infections, autoimmune disorders or psychoneuroimmunology of autoimmune disorders.

25. The composition of claim 1, further comprising DNA-intercalating agents.

26. The composition of claim 1, further comprising agents that block activity of topoisomerase I and topoisomerase II.

27. The composition of claim 1, further comprising cytokines, antimicrobial agents, antiviral agents, hormones, sedatives, keratinocyte growth factor, interleukins, hypnotics, tranquilizers, avitamins, minerals, zinc, dietary supplements, pharmaceutically acceptable stabilizers, adjuvants, diluents and mixtures thereof.

28. A method of treating subjects inflected with COVID-19, the method comprising the step of administering to a subject in need thereof thymus gland hormones.

29. The method of claim 28, wherein the method can also be employed as a preventing measure in healthy subjects in case of exposure to COVID-19.

30. The method of claim 1, wherein the method can also be employed in subjects where thymectomy was performed, including stem cells and thymus bioengineering.

31. The method of claim 28, wherein the thymus gland hormones enhance the endogenous regeneration of the thymus gland via the process of thymopoiesis, thereby promoting the production of mature T cells.

32. The method of claim 28, wherein the thymus gland hormones can be those, but not limited to, hormones secreted by the thymus gland, a combination of them, their active compounds, their progenitors, and their stimulating and mediated compounds and their mixtures thereof.

33. The method of claim 28, wherein the thymus gland hormones are thymosin, thymopoietin, thymulin and thymic humoral factor.

34. The method of claim 28, wherein the thymus gland hormone are found in healthy whole blood or healthy plasma transfusion procured from healthy children where it is permissible by law.

35. The method of claim 28, wherein the thymus gland hormones are found in healthy whole blood or healthy plasma transfusion procured from healthy adults ages 18 to 20 years.

36. The method of claim 28, wherein the pH of healthy children blood or plasma is between 7.35 to 7.45.

37. The method of claim 28, wherein the pH of healthy adults blood or plasma ages 18 to 20 years is between 7.35 to 7.45.

38. The method of claim 28, wherein the thymus gland hormones can be of synthetic nature.

39. The method of claim 28, wherein the human thymus gland hormone is administered in an effective amount equaling to at least to the concentration of the hormone present in children healthy whole blood or healthy plasma or adults healthy whole blood or healthy plasma ages 18 to 20 years.

40. The method of claim 28, wherein the human thymus gland hormone is thymosin administered in a concentration of at least 16.3 μg/ml.

41. The method of claim 28, wherein the human thymus gland hormone is thymopoietin administered in a concentration of at least 1.0 ng/ml.

42. The method of claim 28, wherein the human thymus gland hormone is thymulin administered in a concentration of at least 4.77 log₂.

43. The method of claim 28, wherein the human thymus gland hormone is thymic humoral factor in a concentration of at least 93 nmol/ml.

44. A composition of claim 28, comprising a pharmaceutically acceptable carrier.

45. The composition of claim 28, wherein the pharmaceutically acceptable carrier comprises crystalloids, saline solutions and lactated Ringer's.

46. The composition of claim 28, wherein the pharmaceutically acceptable carrier has a pH range values between 5.5 to 7.45.

47. The method of claim 28, wherein the composition can be administered to a subject via intramuscular (IM), intravenous (IV) intraperitoneal (IP) or intrathecal (IT).

48. The composition of claim 28, further comprising DNA-intercalating agents.

49. The composition of claim 28, further comprising agents that block activity of topoisomerase I and topoisomerase II.

50. The composition of claim 28, further comprising cytokines, antimicrobial agents, antiviral agents, hormones, sedatives, keratinocyte growth factor, interleukins, hypnotics, tranquilizers, avitamins, minerals, zinc, dietary supplements, pharmaceutically acceptable stabilizers, adjuvants, diluents and mixtures thereof.

51. The composition of claim 28, further comprising fenofibrate, EXO-CD24, Allocetra™, hyrdroxychloroquine, remdesivir, ivermectin, tocilizumab, lopinavir, ritonavir, camostat, famotidine, sarilumab, nitazoxanide, corticosteroids, nafamostat, bevacizumab, fluvoxamine, umifenovir, and mixtures thereof.
